(19) United States
(12) Patent Application Publication
(10) Pub. No.: US 2022/0410132 A1
(43) Pub. Date: Dec. 29, 2022

(54) CATALYST AND ITS USE IN ETHYLBENZENE DEALKYLATION

(71) Applicant: SHELL USA, INC., HOUSTON, TX (US)

(72) Inventors: Yuriy Ihorovych YANSON, Rotterdam (NL); Hong Xin LI, Conshohocken, PA (US); Anton PETUSHKOV, Newtown, PA (US); James HUGHES, Malvern, PA (US)

(21) Appl. No.: 17/787,662

(22) PCT Filed: Dec. 21, 2020
(86) PCT No.: PCT/EP2020/087459

§ 371 Date: Jun. 21, 2022

(30) Foreign Application Priority Data
Dec. 23, 2019 (EP) 19219381.1

(51) Int. Cl. B01J 29/40; C07C 4/18

(52) U.S. Cl. CPC B01J 29/40; C07C 4/18; B01J 2229/10; B01J 35/006

(57) ABSTRACT

An ethylbenzene dealkylation catalyst composition comprising a ZSM-5 type zeolite as a carrier component, wherein said zeolite has been synthesized from an aqueous reaction mixture comprising one or more alumina sources, one or more silica sources, one or more alkali sources, and one or more primary and/or secondary amines and wherein the ZSM-5 type zeolite has a number average crystallite size in the range of from 1 to 10 μm and a molar silica-to-alumina ratio (SAR) in the range of from 30 to 70; a method for reducing xylene losses in an ethylbenzene dealkylation process, said method comprising conducting the ethylbenzene dealklylation process in the presence of the afore-mentioned catalyst composition; and a process for the dealkylation of ethylbenzene, which process comprises contacting, in the presence of hydrogen, a feedstock which comprises ethylbenzene with said catalyst composition.

### FIELD OF THE INVENTION

The present invention relates to a catalyst composition containing a ZSM-5 type zeolite and its use in ethylbenzene dealkylation.

### BACKGROUND OF THE INVENTION

Ethylbenzene is one of the aromatic hydrocarbons that is obtained from naphtha pyrolysis or reformate. Reformate is an aromatic product given by the catalysed conversion of straight-run hydrocarbons boiling in the 70 to 190° C. range, such as straight-run naphtha. The catalysts used for the production of reformate are often platinum-on-alumina catalysts. On conversion to reformate, the aromatics content is considerably increased and the resulting hydrocarbon mixture becomes highly desirable as a source of valuable chemicals intermediates and as a component for gasoline. The principle components are a group of aromatics often referred to as BTX: benzene, toluene, and the xylenes, and ethylbenzene. Other components may be present such as their hydrogenated homologues, e.g. cyclohexane.

Of the BTX group, the most valuable components are benzene and the xylenes, and therefore BTX is often subjected to processing to increase the proportion of those two aromatics: hydrodealkylation of toluene to benzene and toluene disproportionation to benzene and xylenes. Within the xylenes, para-xylene is the most useful commodity and xylene isomerisation or transalkylation processes have been developed to increase the proportion of para-xylene.

A further process that can be applied is the hydrodealkylation of ethylbenzene to benzene.

Generally, it is preferred to isolate BTX from the reformate stream, then isolate the C₈ aromatics by distillation, followed by extraction of para-xylene via selective adsorption or crystallisation. The para-xylene lean C₈ aromatics stream is then subjected to xylene isomerisation with the aim of maximising the para-xylene component to be able to recycle the stream and extract more para-xylene. To avoid build-up of ethylbenzene in the recycle stream, ethylbenzene has to be converted. Typically, this is done either by dealkylating ethylbenzene to generate valuable benzene or by reforming ethylbenzene to xylenes to increase the yield of xylenes. In practice, catalyst systems are used to isomerize the xylenes to equilibrium and simultaneously either reform ethylbenzene to xylenes or dealkylate ethylbenzene. The latter process is the subject of the present invention.

In ethylbenzene dealkylation, it is a primary concern to ensure not just a high degree of conversion of ethylbenzene to benzene and isomerise the xylenes close to equilibrium, but also to avoid xylene loss.

Xylenes may typically be lost due to transalkylation, e.g. between benzene and xylene to give toluene, or by addition of hydrogen to form, for example, alkenes or alkanes. A further route for xylene loss is the disproportionation of two xylene molecules, leading to the formation of the significantly less valuable trimethylbenzene (TMB) and toluene.

It is therefore the aim of the present invention to provide a catalyst that will convert ethylbenzene to benzene with a reduced xylene loss, and in particular, with a reduced TMB make.

For the conversion of BTX streams to increase the proportion of closely configured molecules, a wide range of proposals utilizing zeolitic catalysts have been made. One common zeolite group utilized in the dealkylation of ethylbenzene is the MFI zeolites and, in particular, ZSM-5. The ZSM-5 zeolite is well known and documented in the art.

Many preparation routes have been proposed that provide active MFI zeolites, including ZSM-5, see for example U.S. Pat. No. 3,702,886 A, references provided in the Atlas, or Database, of Zeolite Structures, and in other literature references such as by Yu et al. in Microporous and Mesoporous Materials 95 (2006) 234 to 240, and Iwayama et al in U.S. Pat. No. 4,511,547 A.

U.S. Pat. No. 3,702,886 A prepares the zeolites utilizing a silica source, an alumina source and alkali sources and describes the use of a tetraalkylammonium cation, such as tetrapropylammonium (TPA) cation, as an organic structure-directing agent in the preparation of a ZSM-5.

U.S. Pat. No. 8,574,542 B2 describes the preparation of ZSM-5 by synthesis from an aqueous reaction mixture comprising an alumina source, a silica source, an alkali source and L-tartaric acid or a water-soluble salt thereof and the use of said ZSM-5 in a process for the conversion of an aromatic hydrocarbon-containing feedstock, in particular for the selective dealkylation of ethylbenzene.

U.S. Pat. No. 4,312,790 A discloses a method of preparing a noble metal containing zeolite catalyst for use in aromatics processing, in particular xylene isomerization. Said method comprises incorporating a noble metal in a cationic form with a zeolite after crystallization, prior to final catalyst particle formation and prior to any calcination or steaming of said zeolite, said zeolite being characterised by a silica to alumina mole ratio of at least 12 and a Constraint Index in the approximate range of 1 to 12. Example 5 in U.S. Pat. No. 4,312,790 A describes the preparation of a Pt-ZSM-5 catalyst using an alumina binder. The ZSM-5 zeolite in Example 5 was prepared using a mixture comprising tetrapropylammonium (TPA) bromide as the structure-directing agent. Said structure-directing agent was formed in situ using a solution comprising n-propyl bromide and tri-n-propylamine. Said zeolite was mixed with alumina binder and impregnated with platinum prior to extrusion of the Pt-ZSM-5/Al₂O₃ catalyst pellets.

WO 2011/143031 A2 discloses a process for dealkylating ethylbenzene comprising passing a stream comprising ethylbenzene over an effective amount of a catalyst, wherein said catalyst comprises (a) a molecular sieve comprising one or more crystals wherein the molecular sieve has an external surface area of no more than 20 m²/g; and (b) a binder. Preferably, the external surface of the molecular sieve is no more than 12 m²/g, more preferably no more than 8 m²/g. In WO 2011/143031 A2, the molecular sieve may be an MFI zeolite.

Examples in WO 2011/143031 A2 describe the preparation of MFI zeolites using sodium aluminate, silica and n-butylamine as a templating agent. The zeolites prepared had either large crystals (>10 μm) and a high molar silica-to-alumina ratio (SAR) of >75 or small crystals (<1 μm) and a low SAR of <60.

### SUMMARY OF THE INVENTION

It has now been found in the present invention that by producing ZSM-5 crystals having certain molar silica-to-alumina ratios (SAR) and number average crystallite sizes, which are also made using certain compounds as structure-directing agents, it is possible to prepare catalyst compositions that provide significantly reduced xylene losses in ethylbenzene dealkylation.

Accordingly, the present invention provides an ethylbenzene dealkylation catalyst composition comprising a ZSM-5 type zeolite as a carrier component, wherein said zeolite has been synthesized from an aqueous reaction mixture comprising one or more alumina sources, one or more silica sources, one or more alkali sources, and one or more primary and/or secondary amines and wherein the ZSM-5 type zeolite has a number average crystallite size in the range of from 1 to 10 μm and a molar silica-to-alumina ratio (SAR) in the range of from 30 to 70.

The present invention further provides a method for reducing xylene losses in an ethylbenzene dealkylation process, said method comprising conducting the ethylbenzene dealklylation process in the presence of the afore-mentioned catalyst composition.

Also provided by the present invention is a process for the dealkylation of ethylbenzene, which process comprises contacting, in the presence of hydrogen, a feedstock which comprises ethylbenzene with said catalyst composition.

### DETAILED DESCRIPTION OF THE INVENTION

The ZSM-5 type zeolites prepared as described herein have been surprisingly found to provide much reduced xylene losses compared with ZSM-5 type zeolites prepared using other structure-directing agents such as tetrapropylammonium (TPA) compounds. In particular, catalyst compositions comprising ZSM-5 type zeolites prepared and having the characteristics as described herein have been found to result in lower TMB make when used in the dealkylation of ethylbenzene. In addition, it has also been found that said catalysts show surprising additional advantages when the carriers therein are also subjected to a surface modification treatment.

In zeolite characterization, the molar ratio of silica to alumina (SiO₂/Al₂O₃, herein ‘SAR’) is often an important parameter. This parameter is inversely related to the acid site density associated with the presence of aluminium in the framework of a crystalline aluminosilicate zeolite. Conventionally, SAR is determined for crystalline aluminosilicate zeolitic materials by bulk elemental analysis.

The ZSM-5 type zeolite in the present invention has a molar silica-to-alumina ratio (SAR) in the range of from 30 to 70, preferably in the range of from 45 to 70, more preferably in the range of from 45 to 65 and even more preferably in the range of from 45 to 60. This (bulk or overall) SAR can be determined by any one of a number of chemical analysis techniques. Such techniques include X-ray fluorescence, atomic adsorption, and inductive coupled plasma-atomic emission spectroscopy (ICP-AES). All will provide substantially the same bulk ratio value. The molar silica to alumina ratio for use in the present invention is preferably determined by X-ray fluorescence.

The ZSM-5 type zeolite in the present invention can have various particle sizes. Said zeolite has a number average particle diameter (hereinafter referred to as “crystallite size”) in the range of from 1 to 10 μm (micron). The number average crystallite size of the ZSM-5 type zeolite is preferably in the range of from 1 to 7 μm, more preferably in the range of from 1 to 5 μm. As used herein, “crystallite size” is measured by Scanning Electron Microscopy (SEM) with the average based on the number average.

In a preferred embodiment of the present invention the ZSM-5 type zeolite has a molar silica-to-alumina ratio (SAR) in the range of from 30 to 70 and a number average crystallite size selected from one of the following preferred combinations:—(i) a SAR in the range of from 30 to 70 and a number average crystallite size in the range of from 1 to 7 μm; (ii) a SAR in the range of from 30 to 70 and a number average crystallite size in the range of from 1 to 5 μm.

In another preferred embodiment of the present invention the ZSM-5 type zeolite has a molar silica-to-alumina ratio (SAR) in the range of from 45 to 70 and a number average crystallite size selected from one of the following preferred combinations:—(i) a SAR in the range of from 45 to 70 and a number average crystallite size in the range of from 1 to 10 μm; (ii) a SAR in the range of from 45 to 70 and a number average crystallite size in the range of from 1 to 7 μm; (iii) a SAR in the range of from 45 to 70 and a number average crystallite size in the range of from 1 to 5 μm.

In a further preferred embodiment of the present invention the ZSM-5 type zeolite has a molar silica-to-alumina ratio (SAR) in the range of from 45 to 65 and a number average crystallite size selected from one of the following preferred combinations:—(i) a SAR in the range of from 45 to 65 and a number average crystallite size in the range of from 1 to 10 μm; (ii) a SAR in the range of from 45 to 65 and a number average crystallite size in the range of from 1 to 7 μm; (iii) a SAR in the range of from 45 to 65 and a number average crystallite size in the range of from 1 to 5 μm.

In another preferred embodiment of the present invention the ZSM-5 type zeolite has a molar silica-to-alumina ratio (SAR) in the range of from 45 to 60 and a number average crystallite size selected from one of the following preferred combinations:—(i) a SAR in the range of from 45 to 60 and a number average crystallite size in the range of from 1 to 10 μm; (ii) a SAR in the range of from 45 to 60 and a number average crystallite size in the range of from 1 to 7 μm; (iii) a SAR in the range of from 45 to 60 and a number average crystallite size in the range of from 1 to 5 μm.

The ZSM-5 type zeolite used in the ethylbenzene dealkylation catalyst composition of the present invention preferably has a total surface area of greater than 350 m²/g, more preferably greater than 375 m²/g and most preferably greater than 400 m²/g, as measured by ASTM D4365-95.

The ZSM-5 type zeolite used in the ethylbenzene dealkylation catalyst composition of the present invention is synthesized from an aqueous reaction mixture comprising one or more alumina sources, one or more silica sources, one or more alkali sources, and one or more primary and/or secondary amines.

In the present invention, the one or more silica sources are preferably selected from silica sol, silica gel, silica aerogel, silica hydrogel, silicic acid, silicate ester and sodium silicate.

As the alumina source, there may be used known alumina sources which have heretofore been used in the preparation of zeolites, such as sodium aluminate, aluminium sulfate, aluminium nitrate, alumina sol, alumina gel, activated alumina, gamma.-alumina and alpha.-alumina.

Examples of the alkali source are sodium hydroxide and potassium hydroxide, of which sodium hydroxide is preferred. It will be appreciated that if sodium silicate is used as the silica source and sodium aluminate as the alumina source, then both compounds will also serve as the alkali source.

In a particularly preferred embodiment of the present invention, the one or more amines are primary and/or secondary amines having the formulas R¹NH₂ and/or R²R³NH, wherein each of R¹, R², R³ are independently selected from alkyl groups having from 3 to 8 carbon atoms, and where R² and R³ may be the same or different. Examples of preferred amines include propylamine, n-butylamine, n-pentylamine, n-hexylamine, n-heptylamine, n-octylamine, dipropylamine and diisopropylamine.

More particularly, the one or more amines are primary and/or secondary amines having the formulas R¹NH₂ and/or R²R³NH, wherein each of R¹, R², R³ are independently selected from linear alkyl groups having from 3 to 8 carbon atoms, more preferably from 4 to 8 carbon atoms, and where R² and R³ may be the same or different. Examples of preferred linear alkyl amines include n-butylamine, n-pentylamine, n-hexylamine, n-heptylamine, n-octylamine.

In addition to the ZSM-5 type zeolite as hereinbefore described, the catalyst composition according to the present invention preferably further comprises one or more metals and one or more inorganic oxide binders.

In the catalyst composition of the present invention, the ZSM-5 type zeolite can exist in various forms depending on the ion present at the cation sites in the zeolite structure. Generally, the available forms contain an alkali metal ion, an alkaline earth metal ion, or a hydrogen or hydrogen precursor ion at the cation site. In the catalyst composition of the present invention, the zeolite is typically present in the form containing hydrogen or hydrogen precursor; this form is commonly known as the H⁺ form. The zeolite may be used either in a template-free or a template-containing form.

The inorganic oxide binder is preferably a refractory oxide selected from the group consisting of silica, zirconia and titania.

Most preferably, silica is used as the binder in the catalyst composition of the present invention and may be a naturally occurring silica or may be in the form of a gelatinous precipitate, sol or gel. The form of silica is not limited and the silica may be in any of its various forms: crystalline silica, vitreous silica or amorphous silica. The term amorphous silica encompasses the wet process types, including precipitated silicas and silica gels, of pyrogenic or fumed silicas. Silica sols or colloidal silicas are non-settling dispersions of amorphous silicas in a liquid, usually water, typically stabilised by anions, cations, or non-ionic materials.

The silica binder is preferably a mixture of two silica types, most preferably a mixture of a powder form silica and a silica sol. Conveniently powder form silica has a surface area in the range of from 50 to 1000 m²/g; and a mean particle size in the range of from 2 nm to 200 μm, preferably in the range from 2 to 100 μm, more preferably 2-60 μm especially 2-10 μm as measured by

ASTM C 690-1992 or ISO 8130-1. A very suitable powder form silica material is “Sipernat 50”, a white silica powder having predominately spherical particles, available from Evonik (“Sipernat” is a trade name). A very suitable silica sol is that sold under the trade name of “Bindzil” by Nouryon. Where the mixture comprises a powder form silica and a silica sol, then the two components may be present in a weight ratio of powder form to sol in the range of from 1:1 to 10:1, preferably from 2:1 to 5:1, more preferably from 2:1 to 3:1. The binder may also consist essentially of just the powder form silica.

Where a powder form of silica is used as the binder in the catalyst composition of the present invention, preferably a small particulate form is utilised, which has a mean particle size in the range of from 2 to 10 μm as measured by ASTM C 690-1992. An additional improvement in carrier strength can be found with such materials. A very suitable small particulate form is that available from Evonik under the trade name “Sipernat 500LS”.

The silica component used may be pure silica and not as a component in another inorganic oxide. For certain embodiments, the silica and indeed, the carrier, is essentially free of any other inorganic oxide binder material, and especially is free of alumina. Optionally, at most 2 wt % alumina, based on the total carrier, is present.

The carrier in the catalyst composition of the present invention may be considered to be a composite comprising the ZSM-5 type zeolite and the inorganic oxide binder. Said carrier preferably comprises in the range of from 20 to 75 wt % of binder in combination with in the range of from 25 to 80 wt % of the ZSM-5 type zeolite, more preferably in the range of from 20 to 65 wt % of binder in combination with in the range of from 35 to 80 wt % of the ZSM-5 type zeolite, more specifically in the range of from 25 to 60 wt % of binder in combination with in the range of from 40 to 75 wt % of the ZSM-5 type zeolite, even more specifically in the range of from 25 to 55 wt % of binder in combination with in the range of from 45 to 75 wt % of the ZSM-5 type zeolite, most specifically in the range of from 30 to 50 wt % of binder in combination with in the range of from 50 to 70 wt % of the ZSM-5 type zeolite, based on the total weight of the carrier composition. The binder is preferably silica.

The carrier and resulting catalyst composition can contain one or more further zeolites in addition to the afore-mentioned ZSM-5 type zeolite. Preferred further zeolites may be chosen from the group consisting of (other) ZSM-5, ZSM-11, ZSM-12, EU-1, ZSM-57, ZSM-22, ZSM-23, ITQ-1, PSH-3, stilbite, TNU-10, TS-1 and mordenite. Most preferably, the additional zeolite is chosen from the group consisting of ZSM-11, ZSM-12, EU-1 and mordenite. Preferably, the one or more further zeolites are present in the carrier in an amount in the range of from 0 to 35 wt %, based on the total weight of carrier, more preferably in an amount in the range of from 1 to 20 wt %, more preferably in an amount in the range of from 2 to 10 wt %.

In another embodiment, the present invention provides a method for making the afore-mentioned ethylbenzene dealkylation catalyst composition, said method comprising:—

(i) preparing a ZSM-5 type zeolite as a carrier component from an aqueous reaction mixture comprising one or more alumina sources, one or more silica sources, one or more alkali sources, and one or more primary and/or secondary amines;
(ii) preparing a carrier comprising said ZSM-5 type zeolite and one or more inorganic oxide binders; and
(iii) depositing one or more metals on the carrier.

The mixture of ZSM-5 type zeolite and inorganic oxide binders may be shaped into any convenient form such as powders, extrudates, pills and granules. Preference is given to shaping by extrusion. To prepare extrudates, commonly the zeolite will be combined with the binder, preferably silica, and if necessary, a peptizing agent, and mixed to form a dough or thick paste. The peptizing agent may be any material that will change the pH of the mixture sufficiently to induce deagglomeration of the solid particles. Peptizing agents are well known and encompass organic and inorganic acids, such as nitric acid, and alkaline materials such as ammonia, ammonium hydroxide, alkali metal hydroxides, preferably sodium hydroxide and potassium hydroxide, alkali earth hydroxides and organic amines, e.g. methylamine and ethylamine. Ammonia is a preferred peptizing agent and may be provided in any suitable form, for example via an ammonia precursor. Examples of ammonia precursors are ammonium hydroxide and urea. It is also possible for the ammonia to be present as part of the silica component, particularly where a silica sol is used, though additional ammonia may still be needed to impart the appropriate pH change. The amount of ammonia present during extrusion has been found to affect the pore structure of the extrudates which may provide advantageous properties. Suitably the amount of ammonia present during extrusion may be in the range of from 0 to 5 wt % based on the total dry mixture, preferably 0 to 3 wt %, more preferably 0 to 1.9 wt %, on dry basis.

The carrier is conveniently a shaped carrier and may be treated to enhance the activity of the ZSM-5 type zeolite component. Indeed, in a particular embodiment of the present invention, it has been surprisingly found that the catalyst composition of the present invention demonstrates additional performance benefits when the carrier therein has also been subjected to a surface modification treatment.

Thus, in certain embodiments, a surface modification treatment may be performed on the carrier comprising the afore-mentioned ZSM-5 type zeolite prior to impregnation with one or more metals to prepare the catalyst composition of the present invention.

Hence, the present invention also provides a method for making the afore-mentioned ethylbenzene dealkylation catalyst composition, said method comprising:—

(i) preparing a ZSM-5 type zeolite as a carrier component from an aqueous reaction mixture comprising one or more alumina sources, one or more silica sources, one or more alkali sources, and one or more primary and/or secondary amines;
(ii) preparing a carrier comprising said ZSM-5 type zeolite and one or more inorganic oxide binders;
(iii) conducting a surface modification treatment on the ZSM-5 type zeolite; and
(iv) depositing one or more metals on the carrier.

Surface modification of the zeolite reduces the mole percentage of alumina which basically implies that the number of acid sites is reduced. This can be achieved in various ways. A first way is applying a coating of a low acidity inorganic refractory oxide onto the surface of the crystallites of the ZSM-5 type zeolite.

Another very useful way of modifying the surface of the ZSM-5 type zeolite is by subjecting it to a dealumination treatment, for example, such as that described in U.S. Pat. No. 6,949,181 B2.

The surface modification treatment may be conducted on the ZSM-5 type zeolite prior to incorporation in the carrier or it may be performed on the ZSM-5 type zeolite after it is been incorporated into the carrier.

In the present invention, it has been found to be particularly advantageous to perform a dealumination treatment on the carrier comprising a ZSM-5 type zeolite as a carrier component.

Accordingly, preferably the surface modification treatment in the above process for making the afore-mentioned ethylbenzene dealkylation catalyst composition comprises conducting a dealumination treatment on the carrier, either before or after deposition of the one or more metals. Most preferably, a dealumination treatment is conducted on the carrier prior to deposition of the one or more metals.

The dealuminated ZSM-5 type zeolite will have a lower concentration of alumina at the surface than a corresponding ZSM-5 type zeolite which has not been dealuminated. Dealumination can be carried out either on the zeolite per se or on zeolite which has been incorporated into carrier extrudates. In many cases, it is preferred to dealuminate the carrier extrudates. Carrier extrusion may take place either before or after deposition of the one or more metals.

In general, dealumination of the crystallites of a molecular sieve such as a zeolite refers to a treatment, whereby aluminium atoms are either withdrawn from the molecular sieve framework leaving a defect or are withdrawn and replaced by other atoms, such as silicon, titanium, boron, germanium, or zirconium. Removing alumina from zeolite can be carried out in any way known to someone skilled in the art.

Examples of dealumination treatments include steaming, treatment with F-containing salts and treatment with acids such as hydrochloric acid (HCl), nitric acid (HNO₃) or ethylenediamine tetraacetic acid (EDTA).

In U.S. Pat. No. 5,242,676 A, a very suitable method for the dealumination of the surface of zeolite crystallites is disclosed. Another method for obtaining a zeolite having a dealuminated outer surface is disclosed in U.S. Pat. No. 4,088,605 A.

In one embodiment of the present invention, it is preferred to treat the ZSM-5 zeolite particles or carrier extrudate by a steaming process comprising a heat treatment at temperatures above 300° C. in the presence of steam in order to remove alumina from the zeolite framework. The extent of dealumination depends on the steam concentration and the temperature. In a preferred embodiment, the temperature is in the range of from 500 to 750° C. and the steam concentration in air is in the range of from 10 to 25%.

In another embodiment of the present invention, it is preferred to treat the zeolite particles, optionally in combination with binder as a carrier, with a fluorine-containing salt. Most preferably, the dealumination is performed by a process in which the zeolite is contacted with a solution of ammonium fluoride, more specifically a compound chosen from the group consisting of fluorosilicates and fluorotitanates, most preferably a compound chosen from the group of fluorosilicates. These processes are described in more detail in U.S. Pat. No. 4,753,910 A.

Most preferably, the dealumination process comprises contacting the zeolite with a solution of a fluorosilicate salt wherein the fluorosilicate salt is represented by the formula:

(A)_{2/b}SiF₆

wherein ‘A’ is a metallic or non-metallic cation other than H+ having the valence ‘b’. Examples of cations ‘b’ are alkylammonium, NH₄⁺, Mg⁺⁺, Li⁺, Na⁺, K⁺, Ba⁺⁺, Cd⁺⁺, Cu⁺, Ca⁺⁺, Cs⁺, Fe⁺⁺, Co⁺⁺, Pb⁺⁺, Mn⁺⁺, Rb⁺, Ag⁺, Sr⁺⁺, Tl⁺, and Zn⁺⁺. Preferably ‘A’ is the ammonium cation.

The solution comprising the fluorosilicate salt preferably is an aqueous solution. The concentration of the salt preferably is at least 0.005 mole of fluorosilicate salt/l, more preferably at least 0.007, most preferably at least 0.01 mole of fluorosilicate salt/l. The concentration preferably is at most 0.5 mole of fluorosilicate salt/l, more preferably at most 0.3, most preferably at most 0.1 of fluorosilicate salt/l. Preferably, the weight ratio of fluorosilicate salt solution to zeolite is from 50:1 to 1:4 of fluorosilicate solution to zeolite. If the zeolite is present together with binder, the binder is not taken into account for these weight ratios.

The pH of the aqueous fluorosilicate containing solution preferably is between 2 and 8, more preferably between 3 and 7.

The zeolite material preferably is contacted with the fluorosilicate salt solution for a period of from 0.5 to 20 hours, more specifically of from 1 to 10 hours. The temperature preferably is of from 10 to 120° C., more specifically of from 20 to 100° C. The amount of fluorosilicate salt preferably is at least 0.002 moles of fluorosilicate salt per 100 grams of total amount of zeolite, more specifically at least 0.003, more specifically at least 0.004, more specifically at least 0.005 moles of fluorosilicate salt per 100 grams of total amount of zeolite. The amount preferably is at most 0.5 moles of fluorosilicate salt per 100 grams of total amount of zeolite, more preferably at most 0.3, more preferably at most 0.1 moles of fluorosilicate salt per 100 grams of total amount of zeolite. If the zeolite is present together with binder, the binder is not taken into account for these weight ratios.

Of the (surface) dealumination methods described above, the method involving the treatment with a hexafluorosilicate, most suitably ammoniumhexafluorosilicate (AHS) as described in U.S. Pat. No. 6,949,181 B2, is the most preferred in the process for making the afore-mentioned ethylbenzene dealkylation catalyst composition of the present invention. Preferably the concentration of ammoniumhexafluorosilicate (AHS) is in the range of from 0.005 to 0.5M. Preferably the concentration is in the range of from 0.01 to 0.2M, more preferably 0.01 to 0.05M, and especially 0.01 to 0.03M, which has been found to provide an advantageous catalyst composition.

The one or more metals in the catalyst composition of the present invention are preferably those comprising metals selected from Groups 6, 7, 8, 9, 10 and 14 of the Periodic Table (as defined in IUPAC Periodic Table of Elements dated 1 May 2013). More preferably, the one or more metals in the catalyst composition of the present invention are selected from those comprising chromium, ruthenium, rhenium, iron, chromium, molybdenum, tungsten, palladium, platinum, tin, lead, silver, copper, and nickel.

Most preferably, the catalyst composition of the present invention comprises platinum as a catalytically active metal. Optionally, the catalyst composition of the present invention comprises platinum as a catalytically active metal and one or more additional metal promoters selected from tin, lead, copper, nickel, gallium, cerium and silver.

The weight amounts of the one or more metals are calculated, based on total weight of catalyst composition and independent of the actual form of the metal.

The amount of said one or more metals in the catalyst composition depends on the nature of the metal employed. For example, oxidic or sulphidic hydrogenation metals (i.e. chromium, molybdenum, tungsten and iron) may be typically utilised in amounts above 1 wt %, calculated as amount of said metals, based on total weight of catalyst composition and independent of the actual form of the metal. In contrast, other metals (for example, rhenium, ruthenium, platinum and palladium) may be conveniently employed in amounts less than 1 wt %, calculated as amount of said metals, based on total weight of catalyst composition and independent of the actual form of the metal.

In a preferred embodiment of the catalyst composition of the present invention, platinum is present as a catalytically active metal in an amount in the range of from 0.001 to 0.1 wt %, based on total weight of the catalyst composition. Most suitably, platinum is present as a catalytically active metal in an amount in the range of from 0.01 to 0.1 wt %, preferably from 0.01 to 0.05 wt %, based on total weight of the catalyst composition.

Optionally, in addition to platinum, one or more additional metals selected from tin, lead, copper, nickel, and silver are present in the catalyst composition in an individual amount of less than 1 wt %, based on total weight of the catalyst composition. The optional one or more additional metals are most suitably present in an individual amount in the range from 0.0001 to 0.5 wt %, preferably in an amount in the range of from 0.01 to 0.5 wt %, more preferably in an amount in the range of from 0.1 to 0.5 wt %, based on total weight of the catalyst composition. If tin or lead is the additional metal, then it is present in an amount in the range of from 0.01 to 0.5 wt %, based on total catalyst, most suitably present in an amount in the range of from 0.1 to 0.5 wt %, preferably 0.2 to 0.5 wt %, based on total weight of the catalyst composition.

The catalyst composition of the present invention may be prepared using standard techniques for combining the ZSM-5 type zeolite, binder, and optional other carrier components; optionally, shaping; impregnating with the one or more catalytically active metal compounds; and any subsequent useful process steps such as shaping (if not carried out prior to impregnation), drying, calcining, and reducing.

The metals emplacement onto the formed carrier may be by methods usual in the art. The metals can be deposited onto the carrier materials prior to shaping, but it is preferred to deposit them onto a shaped carrier.

It is preferable that a calcination step be carried out on the resultant extrudate prior to emplacement of the metals, this is preferably carried out at temperatures above 500° C. and typically above 600° C.

Pore volume impregnation of the metals from a metal salt solution is a very suitable method of metals emplacement onto a shaped carrier. The metal salt solutions may have a pH in the range of from 1 to 12. The platinum salts that may conveniently be used are chloroplatinic acid and ammonium stabilised platinum salts. An additional silver, nickel or copper metal salt may be added in the form of water soluble organic or inorganic salt in solution. Examples of suitable salts are nitrates, sulphates, hydroxides and ammonium (amine) complexes. Examples of suitable tin salts that may be utilized are stannous (II) chloride, stannic (IV) chloride, stannous sulphate, and stannous acetate. Examples of suitable lead salts are lead acetate, lead nitrate, and lead sulphate.

Where there is more than one metal in the catalyst composition of the present invention, the metals may be impregnated either sequentially or simultaneously. It is preferable that the metals be added simultaneously. Where simultaneous impregnation is utilised, the metal salts used must be compatible and not hinder the deposition of the metals.

After shaping of the carrier, and also after impregnation of the one or more metals, the carrier/catalyst composition is suitably dried, and calcined. Drying temperatures are suitably 50 to 200° C.; drying times are suitably from 0.5 to 5 hours. Calcination temperatures are very suitably in the range of from 200 to 800° C., preferably 300 to 600° C., most preferably, the calcination temperature is of from 400 to 475° C. For calcination of the carrier, a relatively short time period is required, for example 0.5 to 3 hours. For calcination of the catalyst composition, it may be necessary to employ controlled temperature ramping at a low rate of heating to ensure the optimum dispersion of the metals: such calcination may require from 5 to 20 hours.

Prior to use, it is generally necessary to ensure that any hydrogenation metals on the catalyst composition are in metallic (and not oxidic) form. Accordingly, it is useful to subject the catalyst composition of the present invention to reducing conditions, which are, for example, heating in a reducing atmosphere, such as in hydrogen optionally diluted with an inert gas, or mixture of inert gases, such as nitrogen and carbon dioxide, at a temperature in the range of from 150 to 600° C. for from 0.5 to 5 hours.

The catalyst composition of the present invention finds particular use in the selective dealkylation of ethylbenzene.

The ethylbenzene feedstock most suitably originates from a reforming unit or naphtha pyrolysis unit or is the effluent of a xylene isomerisation or transalkylation unit. After distillation and para-xylene extraction, such feedstock usually comprises C₇ to C₉ hydrocarbons and, in particular, one or more of o-xylene, m-xylene, and p-xylene, in addition to ethylbenzene. Generally, the amount of ethylbenzene in the feedstock is in the range of from 0.1 to 50 wt % and the total xylene content is typically at least 20 wt %. Typically, the xylenes will not be in a thermodynamic equilibrium, and the content of p-xylene will accordingly be lower than that of the other isomers.

The feedstock is contacted with the catalyst composition of the present invention in the presence of hydrogen. This may be carried out in a fixed bed system. Such a system may be operated continuously or in batch fashion. Preference is given to continuous operation in a fixed bed system. The catalyst may be used in one reactor or in several separate reactors in series or operated in a swing system to ensure continuous operation during catalyst change-out.

The dealkylation process is suitably carried out at a temperature in the range of from 300 to 500° C., a pressure in the range of from 0.1 to 50 bar (10 to 5,000 kPa), using a liquid hourly space velocity of in the range of from 0.5 to 20 h⁻¹. A partial pressure of hydrogen in the range of from 0.05 to 30 bar (5 to 3,000 kPa) is generally used. The hydrogen to feed molar ratio is in the range of from 0.5 to 100, generally from 1 to 10 mol/mol.

The following Examples illustrate the present invention.

### EXAMPLES

### Zeolite Preparation

### Zeolite A (Comparative)

536 grams of colloidal silica (Nyacol, 40 wt % SiO₂), 25.4 grams of sodium aluminate (43 wt % solution), 28.5 grams of tetrapropylammonium (TPA) bromide (50 wt % solution), 7.8 grams of tetramethylammonium (TMA) chloride solution (50 wt % solution), 3.1 grams of sodium hydroxide (50 wt % solution) and 353 grams of water were mixed together. The gel was crystallized at 170° C. for 24 hours.

The crystalline product was filtered, washed with deionized water and dried in air. The zeolite powder was calcined at 550° C. for 6 hours to remove the organic molecules from the pores. The product was analyzed by powder XRD and was shown to be pure phase ZSM-5 (MFI). Said zeolite had a SAR of 62. The crystal size was analyzed by SEM and the average crystal size was shown to be 2.3 micron.

### Zeolite B (Comparative)

780 grams of colloidal silica (Nyacol, 40 wt % SiO₂), 44.4 grams of sodium aluminate (43 wt % solution), 41.5 grams of tetrapropylammonium (TPA) bromide (50 wt % solution), 1 gram of sodium hydroxide (50 wt % solution) and 517 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 51. The crystal size was analyzed by SEM and the average crystal size was shown to be 0.8 micron.

### Zeolite C

687 grams of colloidal silica (Nyacol, 40 wt % SiO₂), 35.5 grams of sodium aluminate (43 wt % solution), 16.9 grams of 1-butylamine, 17.1 grams of sodium hydroxide (50 wt % solution) and 641 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 55. The crystal size was analyzed by SEM and the average crystal size was shown to be 2.9 micron.

### Zeolite D

715 grams of colloidal silica (Nyacol, 40 wt % SiO₂), 36.9 grams of sodium aluminate (43 wt % solution), 20.7 grams of 1-pentylamine, 17.8 grams of sodium hydroxide (50 wt % solution) and 667 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 50. The crystal size was analyzed by SEM and the average crystal size was shown to be 2.3 micron.

### Zeolite E

700 grams of colloidal silica (Nyacol, 40 wt % SiO₂), 36.2 grams of sodium aluminate (43 wt % solution), 23.6 grams of 1-hexylamine, 17.4 grams of sodium hydroxide (50 wt % solution) and 653 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 49. The crystal size was analyzed by SEM and the average crystal size was shown to be 3.8 micron.

### Zeolite F

720 grams of colloidal silica (Nyacol, 40 wt % SiO₂), 37.2 grams of sodium aluminate (43 wt % solution), 27.6 grams of 1-heptylamine, 17.9 grams of sodium hydroxide (50 wt % solution) and 672 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 50. The crystal size was analyzed by SEM and the average crystal size was shown to be 4.4 micron.

### Zeolite G

709 grams of colloidal silica (Nyacol, 40 wt % SiO₂), 36.6 grams of sodium aluminate (43 wt % solution), 30.5 grams of 1-octylamine, 17.6 grams of sodium hydroxide (50 wt % solution) and 661 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 50. The crystal size was analyzed by SEM and the average crystal size was shown to be 4.0 micron.

### Zeolite H

709 grams of colloidal silica (Nyacol, 40 wt % SiO₂), 36.2 grams of sodium aluminate (43 wt % solution), 23.6 grams of dipropylamine, 17.4 grams of sodium hydroxide (50 wt % solution) and 653 grams of water were mixed together. The gel was crystallized at 180° C. for 18 hours.

The crystalline product was filtered, washed with deionized water and dried in air. The zeolite powder was calcined at 550° C. for 6 hours to remove the organic molecules from the pores.

The product was analyzed by powder XRD and was shown to be pure phase ZSM-5 (MFI). Said zeolite had a SAR of 47. The crystal size was analyzed by SEM and the average crystal size was shown to be 2.6 micron.

### Catalyst Preparation

Catalysts A-H were prepared from the zeolite samples A-H by mixing the ZSM-5 zeolite with silica as binder, kneading and extruding to form a shaped carrier, and then impregnating with hydrogenation metal by pore volume impregnation. Each carrier contained 60 wt % zeolite bound with 40 wt % silica binder (a mixture of “Sipernat 50” from Evonik and “Bindzil 30NH3” silica sol from Nouryon in a weight ratio of approximately 2:1). The extrudates were calcined at 500° C., and impregnated with a Pt solution so that the final catalysts each had a composition with 0.02 wt % Pt.

Catalyst I was prepared by mixing, kneading and extruding 60 wt % of commercial ZSM-5 CBV 5524G (Zeolyst,

SAR 50) zeolite with 40 wt % silica binder (a mixture of “Sipernat 50” from Evonik and “Bindzil 30NH3” silica sol from Nouryon in a weight ratio of approximately 2:1). The extrudate was calcined at 500° C. The calcined extrudate was treated with 0.03M ammonium hexafluorosilicate (AHS) solution, and subsequently impregnated with a Pt solution so that the final catalyst had a composition with 0.02 wt % Pt.

Catalysts J-Q were prepared by mixing, kneading and extruding 60 wt % of ZSM-5 zeolite (zeolites A-H, respectively) with 40 wt % silica binder (a mixture of “Sipernat 50” from Evonik and “Bindzil 30NH3” silica sol from Nouryon in a weight ratio of approximately 2:1). The extrudates were calcined at 500° C. The calcined extrudates were treated treated with 0.03M ammonium hexafluorosilicate (AHS) solution, and subsequently impregnated with a Pt solution so that the final catalyst had a composition with 0.02 wt % Pt.

Table 1 below summarises the catalysts prepared.

TABLE 1

| | Zeolite | | | | | |
|---|---|---|---|---|---|---|
| | | | Treatment | | Average | BET Total |
| Catalyst | Zeolite | Templating | with 0.03M | | Crystal Size | Surface Area |
| Designation | Designation | Agent used | AHS*** | SAR | (μm) | (m²/g)**** |
| A (Comp.) | A (Comp.) | TPABr*/TMACl** | No | 62 | 2.3 | 432 |
| B (Comp.) | B (Comp.) | TPABr* | No | 51 | 0.8 | 443 |
| C | C | 1-butylamine | No | 55 | 2.9 | 462 |
| D | D | 1-pentylamine | No | 50 | 2.3 | 434 |
| E | E | 1-hexylamine | No | 49 | 3.8 | 421 |
| F | F | 1-heptylamine | No | 50 | 4.4 | 436 |
| G | G | 1-octylamine | No | 50 | 4.0 | 418 |
| H | H | Dipropylamine | No | 47 | 2.6 | 424 |
| I (Comp.) | I(Comp.) | Not disclosed | Yes | 50 | 0.2 | 425 |
| J (Comp.) | A (Comp.) | TPABr*/TMACl** | Yes | 62 | 2.3 | 432 |
| K (Comp.) | B (Comp.) | TPABr* | Yes | 51 | 0.8 | 443 |
| L | C | 1-butylamine | Yes | 55 | 2.9 | 462 |
| M | D | 1-pentylamine | Yes | 50 | 2.3 | 434 |
| N | E | 1-hexylamine | Yes | 49 | 3.8 | 421 |
| O | F | 1-heptylamine | Yes | 50 | 4.4 | 436 |
| P | G | 1-octylamine | Yes | 50 | 4.0 | 418 |
| Q | H | Dipropylamine | Yes | 47 | 2.6 | 424 |

*Tetrapropylammonium (TPA) bromide.
**Tetramethylammonium (TMA) chloride.
***Zeolite subjected to treatment with 0.03M ammonium hexafluorosilicate (AHS) solution.
****As measured by ASTM D4365-95.

### Catalyst Testing

The catalysts were subjected to a catalytic test that mimics typical industrial application conditions for ethylbenzene dealkylation in a fixed bed reactor unit. The activity test used a feed representative of feeds typically used in industrial units. The composition of the feed used in testing is summarized in Table 2.

TABLE 2
Composition of the feed used in the activity testing

| Feed composition | | |
|---|---|---|
| EB | wt % | 13.1 |
| pX | wt % | 3.7 |
| oX | wt % | 17.8 |
| mX | wt % | 63.8 |
| toluene | wt % | <0.1 |
| benzene | wt % | <0.01 |
| C₇−C₉−paraffins | wt % | 1.5 |
| C₉+ aromatics | wt % | <0.1 |
| Total | wt % | 100.00 |
| C₈ aromatics | sum | |
| EB in C₈ aromatics feed | wt % | 13.3 |
| pX in xylenes in feed | wt % | 3.8 |
| oX in xylenes in feed | wt % | 18.1 |
| mX in xylenes in feed | wt % | 64.8 |

The activity test is performed in a fixed bed unit with online GC analysis once the catalyst is in its reduced state, which was achieved by exposing the dried and calcined catalyst to atmospheric hydrogen (>99% purity) at 450° C. for 1 hour.

After reduction, the reactor is cooled down to 380° C., pressurized to 1.2 MPa and the feed is introduced at a weight hourly space velocity of 12 g feed/g catalyst/hour and a hydrogen to feed ratio of 2.5 mol.mol⁻¹. Subsequently, the temperature is increased to 450° C. and the weight hourly space velocity decreased to 10 g feed/g catalyst/hour and the hydrogen to feed ratio of 1 mol.mol⁻¹. This step contributes to enhanced catalyst aging, and therefore allows comparison of the catalytic performance at stable operation. After 24 hours, the conditions were switched to the actual operating conditions.

In the present case, a weight hourly space velocity of 12 h⁻¹, a hydrogen to feed ratio of 2.5 mol.mol⁻¹, and a total system pressure of 1.3 MPa was used. The temperature was varied between 340 and 380° C. to achieve the required conversion for easier comparison.

The performance characteristics evaluated in this test are as follows:

Ethylbenzene conversion (EB conversion) is the weight percent of ethylbenzene (EB) converted by the catalyst into benzene and ethylene, or other molecules. It is defined as wt. % ethylbenzene in feed minus wt. % ethylbenzene in product divided by wt. % ethylbenzene in feed times 100%.

The formation of C₉ aromatic components, such as trimethylbenzene (TMB) is unwanted as it forms at the expense of preferred products such as p-xylene and benzene.

### Results

Table 3 below shows the performance of the catalysts at 65% ethylbenzene (EB) conversion.

TABLE 3

| | | EB | Reactor | |
|---|---|---|---|---|
| | | conversion | temperature | TMB make |
| Catalyst | Zeolite | (wt %) | (° C.) | (wt %) |
| A | A | 65 | 375 | 0.456 |
| (Comp.) | (Comp.) | | | |
| B | B | 65 | 362 | 0.777 |
| (Comp.) | (Comp.) | | | |
| C | C | 65 | 358 | 0.305 |
| D | D | 65 | 364 | 0.383 |
| E | E | 65 | 362 | 0.349 |
| F | F | 65 | 361 | 0.267 |
| G | G | 65 | 363 | 0.264 |
| H | H | 65 | 356 | 0.222 |
| I | I* | 65 | 376 | 0.725 |
| (Comp.) | (Comp.) | | | |
| J | A* | 65 | 371 | 0.366 |
| (Comp.) | (Comp.) | | | |
| K | B* | 65 | 362 | 0.560 |
| (Comp.) | (Comp.) | | | |
| L | C* | 65 | 360 | 0.234 |
| M | D* | 65 | 363 | 0.274 |
| N | E* | 65 | 360 | 0.219 |
| O | F* | 65 | 361 | 0.168 |
| P | G* | 65 | 360 | 0.144 |
| Q | H* | 65 | 355 | 0.208 |

*Zeolite subjected to treatment with 0.03M AHS.

From the data in Table 3, it is clear that catalysts C-H prepared with ZSM-5 zeolites synthesized with primary and secondary amine templates of different length show significantly lower TMB make than similar catalysts comprising comparable TPA-templated or commercial zeolites (comparative Catalysts A, B and I) at same EB conversion.

Table 3 shows that whilst the TMB make can be improved (i.e. further lowered) for comparative catalysts comprising TPA-templated zeolites (comparative Catalysts A and B) by additionally subjecting zeolites A and B to a selectivation treatment with AHS (comparative Catalysts J and K), the resulting TMB make is generally still greater for treated comparative Catalysts J and K than observed for untreated Catalysts C-G.

Hence, the catalysts of the present invention allow for advantageous reductions in TMB make without the need for additional catalyst treatments.

However, it is also apparent in Table 3 that additionally subjecting zeolites C-H to a selectivation treatment using AHS results in further synergistic improvements in reduced TMB make. Catalysts L-Q according to the present invention demonstrate particularly efficacious selectivity in combination with lower temperatures (i.e. increased catalyst activity) to achieve 65% EB conversion.

1. An ethylbenzene dealkylation catalyst composition comprising a ZSM-5 type zeolite as a carrier component, wherein said zeolite has been synthesised from an aqueous reaction mixture comprising one or more alumina sources, one or more silica sources, one or more alkali sources, and one or more primary and/or secondary amines and wherein the ZSM-5 type zeolite has a number average crystallite size in the range of from 1 to 10 μm and a molar silica-to-alumina ratio (SAR) in the range of from 30 to 70.

2. Catalyst composition according to claim 1, wherein the one or more amines are primary and/or secondary amines having the formulas R¹NH₂ and/or R²R³NH, wherein each of R¹, R², R³ are independently selected from alkyl groups having from 3 to 8 carbon atoms, and where R² and R³ may be the same or different.

3. Catalyst composition according to claim 1, wherein the one or more amines are primary and/or secondary amines having the formulas R¹NH₂ and/or R²R³NH, wherein each of R¹, R², R³ are independently selected from linear alkyl groups having from 3 to 8 carbon atoms, and where R² and R³ may be the same or different.

4. Catalyst composition according to claim 1, wherein the one or more silica sources are selected from silica sol, silica gel, silica aerogel, silica hydrogel, silicic acid, silicate ester and sodium silicate.

5. Catalyst composition according to claim 1, wherein the ZSM-5 type zeolite has a number average crystallite size in the range of from 1 to 7 μm.

6. Catalyst composition according to claim 1, wherein the ZSM-5 type zeolite has a molar silica-to-alumina ratio (SAR) in the range of from 45 to 70.

7. Catalyst composition according to claim 1, wherein the composition further comprises one or more metals and one or more inorganic oxide binders.

8. Catalyst composition according to claim 7, wherein the one or more metals are selected from ruthenium, rhenium, iron, chromium, molybdenum, tungsten, palladium, platinum, tin, lead, silver, copper, and nickel.

9. Catalyst composition according to claim 1, wherein a carrier therein comprising the ZSM-5 type zeolite as a carrier component has been subjected to a dealumination treatment.

10. A method for reducing xylene losses in an ethylbenzene dealkylation process, said method comprising conducting the ethylbenzene dealklylation process in the presence of a catalyst composition according to claim 1.

11. A process for the dealkylation of ethylbenzene, which process comprises contacting, in the presence of hydrogen, a feedstock which comprises ethylbenzene with a catalyst composition as claimed according to claim 1.
